(19) United States
(12) Patent Application Publication
(10) Pub. No.: US 2026/0286234 A1
(43) Pub. Date: Sep. 24, 2026

(54) CONTINUOUS HYDRODEOXYGENATION OF LIGNIN TO JET-RANGE AROMATIC HYDROCARBONS

(71) Applicants: Alliance for Energy Innovation, LLC, Golden, CO (US); MASSACHUSETTS INSTITUTE OF TECHNOLOGY, Cambridge, MA (US)

(72) Inventors: Gregg Tyler BECKHAM, Golden, CO (US); Michael L. STONE, Cambridge, MA (US); Yuriy ROMÁN, Cambridge, MA (US); Matthew S. WEBBER, Cambridge, MA (US)

(21) Appl. No.: 19/100,619

(22) PCT Filed: Aug. 4, 2023
(86) PCT No.: PCT/US2023/029488

§ 371 Date: Feb. 3, 2025

Related U.S. Application Data
(60) Provisional application No. 63/395,067, filed on Aug. 4, 2022.

(51) Int. Cl. C10G 3/00; B01J 8/04

(52) U.S. Cl. CPC C10G 3/50; B01J 8/04; B01J 2208/025; C10G 2300/1014; C10G 2300/70; C10G 2300/80; C10G 2400/08; C10G 2400/30

(57) ABSTRACT

Described herein are systems and methods for the catalytic deoxygenation of lignin to generate low-oxygen aromatics that may be useful as a sustainable aviation or marine fuel. The provided systems and methods may be performed continuously without the need for a solvent, increasing both efficiency and cost effectiveness.

### CROSS-REFERENCE TO RELATED APPLICATIONS

This application claims priority from U.S. Provisional Patent Application No. 63/395,067, filed on Aug. 4, 2022, the contents of which are incorporated herein by reference in their entirety.

### CONTRACTUAL ORIGIN

This invention was made with government support under Contract No. DE-AC36-08GO28308 awarded by the Department of Energy. The government has certain rights in the invention.

### BACKGROUND

In 2019, 106 billion gallons of jet fuel were consumed globally and this number is expected to more than double by 2050 as a result of increased travel demand. To mitigate its impact on climate change, the aviation industry has pledged to reduce net CO₂ emissions in half by 2050. To achieve this goal, sustainable aviation fuel (SAF) must be deployed at a massive scale. Today, most commercially available SAF is synthesized via deoxygenation of plant-derived lipid feedstocks to produce iso- and n-alkane hydrocarbon blends, but these feedstocks are not available in the volumes required to meet the projected fuel demand. Notably, cycloalkanes and aromatics together comprise between 30 and 70 wt % of modern aviation fuel or between 32 and 74 billion gallons per year. However, current SAF technologies cannot produce the aromatic or the cycloalkane components at the scale necessary to satisfy the required properties of jet fuel (namely fuel density and elastomer compatibility) to power the existing fleet of aircrafts, which is a constraint that has resulted in a 50% blending wall with conventional fossil fuels. A technology capable of generating aromatics and cycloalkanes from renewable feedstocks is thus essential for the development of a viable 100% drop-in SAF.

Lignin is the largest source of renewable aromatics available in nature comprising between 15-30% of lignocellulosic biomass. In principle, the 90 million dry-tons of lignin harvested in the United States in 2017 could generate 15 billion gallons of jet-range hydrocarbons based on carbon content. By 2040, this value could increase to 63 billion gallons annually (>50% of current global demand) given projections on future lignin availability. Despite its potential, lignin is currently either re-dispersed in the field or slated to be burned for low grade heat by the cellulosic ethanol and paper industries because lignin becomes highly recalcitrant when separated from the cellulose fibers in biomass. Alternative lignin extraction methods that rely on reductive catalytic fractionation (RCF), protection group chemistries or flow-through solvolysis minimize re-condensation pathways, but the resulting lignin oils have high oxygen contents ranging between 27-34 wt % which must be reduced to trace levels for use in jet fuel. To this end, significant progress has been made in the development of catalysts capable of hydrodeoxygenation (HDO) of oxygenated aromatics. However, the translation of promising catalysts to viable processing strategies with real lignin feedstocks has been hindered by major challenges, such as the use of expensive noble-metal catalysts, catalyst deactivation coupled with low deoxygenation efficiency when using real feedstocks, excessive hydrogen consumption due to ring hydrogenation and hydrocracking, and low carbon yields due to condensation/coking, hydrocracking to gaseous products, or upgrading of only the monomeric fraction.

### SUMMARY

Described herein are systems and methods for the catalytic deoxygenation of lignin to generate low-oxygen aromatics that may be useful as a sustainable aviation or marine fuel. The provided systems and methods may be performed continuously without the need for a solvent, increasing both efficiency and cost effectiveness.

In one aspect, provided is a method for generating aromatic compounds from a lignin feed stock by utilizing a two stage process, first at a lower temperature and second at a higher temperature to fully deoxygenate the lignin while avoiding unwanted secondary reactions such as condensation reactions. This method may utilize a single or multiple catalysts, including as an example an Mo2C-based catalyst. The first, lower temperature may, for example, be selected from the range of 250° C. to 450° C., 300° C. to 400° C., 325° C. to 375° C., 325° C. to 350° C., or about 350° C. The second, higher temperature may, for example, be selected from the range of 275° C. to 475° C., 325° C. to 425° C., 350° C. to 400° C., 350° C. to 375° C., or about 375° C. The resulting aromatic hydrocarbons may have very low oxygen content, for example, having a mole fraction of oxygen in the resulting compounds of less than or equal to 10%, 5%, 1%, 0.5%, or 0.1%.

The method may be performed with neat lignin oil, e.g., lignin without the presence of a solvent. The ability to react neat lignin oil could provide various benefits such as a reduction in cost, smaller reactor requirements, etc. However, the described systems and methods also can be utilized in situations where it is preferable to dissolve the lignin reactant into a solvent, such as an aromatic solvent, for example, toluene.

In an aspect, provide is a system or reactor for the generation of low-oxygen aromatics comprising: a first reaction zone comprising a first catalyst at a first temperature; a second reaction zone comprising a second catalyst at a second temperature; where in the reactor is capable of performing hydrodeoxygenation of lignin to generate aromatic products. The reactor may be a packed bed reactor or a trickle bed reactor. The reactor may also allow for continuous process flow, either as individual steps (e.g., each step is continuous) or a single continuous process. The reactor may be capable of steady state operation.

Other catalysts may be useful including supported metal catalysts as the second, higher-temperature catalyst. Additionally, the described process may be altered to generate cycloalkanes rather than aromatic products. This may be accomplished by incorporating a metal-doped Mo2C-based catalyst or substituting one or both of the catalysts with a catalyst comprising nickel phosphide.

In an aspect, provided is a catalyst comprising Mo2C capable of partially or fully deoxygenating lignin, as described by the systems and methods provided herein.

### BRIEF DESCRIPTION OF DRAWINGS

Some embodiments are illustrated in referenced figures of the drawings. It is intended that the embodiments and figures disclosed herein are to be considered illustrative rather than limiting.

FIG. 1 provides and Overview schematic of HDO of RCF lignin oil. FIG. 1A provides a summary of the lignin monomers quantified as described herein at all levels of partial conversion. Monomer conversion in single pass or two-pass achieves similar results yielding propylbenzene as a major product. The darker shades represent the major reaction pathway on Mo2C and the lighter shades are products resulting from side-reactions, including rearrangement of the functional group position and ring alkylation (products noted with an asterisk), which have previously been observed during deoxygenation with molybdenum oxide catalysts. The acronyms are as follows: PS: propylsyringol, PS=: propenylsyringol, PS—OH: dihydrosinapyl alcohol, PG: propylguaiacol, PG=: propenylguaiacol, PG—OH: dihydroconiferyl alcohol, PP: propylphenol, Alk. PG: methyl-propylguaiacol, Alk. PP: methyl-propylphenol, Rearr. PG: methoxy-propylphenol, PB—OMe: propylanisole, PB: propylbenzene, PCH: propylcyclohexane. FIG. 1B shows examples of dimers identified in the feed, intermediates, and products. Dimer conversion is sensitive to temperature with a parallel pathway toward condensation products at higher temperatures.

FIG. 2 illustrates Catalyst-free control experiments to evaluate lignin oil stability. FIG. 2A shows mass balance on steady state samples collected during no-catalyst control experiments. The bars show mass fractions of monomers and non-monomers, and the product bars are scaled by the overall mass recovery. The height of the bar is the average value, and the error bars show one standard deviation for 3 steady state samples. The feed bar is included for each experiment to account for slight variability between batches of RCF oil. FIG. 2B provides GPC on the feed (“RCF Oil”) and the products from no-catalyst control experiments at different temperatures, with the inset showing the dimer and oligomer portion of the curve to highlight the shift in product distribution with increasing temperature. All samples were prepared to the same concentration of 2 mg/mL and the traces were not normalized or scaled.

FIG. 3 illustrates evaluation of activity and stability of Mo2C for solvent-free lignin HDO. FIG. 3A shows steady state results of monomer distribution and monomer mole balance from HDO experiments flowing neat RCF lignin oil (“Feed”) over a packed bed of 2.88 g Mo2C (60-100 mesh) in a trickle bed reactor. Steady state results were taken after 2.5 h on stream, combined from several hours, and the water formed during reaction was removed prior to performing mass and mole balance calculations. Each bar is labelled with the reactor temperature and the weighted hourly space velocity (WHSV) calculated as the mass flowrate of lignin oil divided by mass of catalyst. The transient distribution of quantified monomers as a function of time on stream at (FIG. 3B) 300° C., (FIG. 3C) 325° C., and (FIG. 3D) 350° C. and 0.1 mL/min lignin oil (WHSV=2.35 h−1). FIG. 3E provides the conversion of oxygen from the monomer fraction for each experiment shown in FIGS. 3B-3D, plotted as the mole percent of oxygen removed from the monomers at each time on stream. Constant conditions: 900 psi, 2.88 g Mo2C (60-100 mesh), 90 mL/min H2, and toluene at 1 mL/min for 30 min during start-up.

FIG. 4 provides optimization of deep deoxygenation of lignin oil for jet-range aromatic production. (FIG. 4A) Carbon, oxygen, and hydrogen content in the oil on a mass basis, as determined via total carbon analysis. The weight percent results of total carbon analysis were multiplied by the mass recovery of oil at steady state after removing the aqueous fraction to generate the plot. (FIG. 4B) Results of quantitative GCxGC analysis of the final deoxygenated oils from each of the experiments shown in FIG. 4A. (FIG. 4C) Simulated distillation generated by the ASTM D2887 method of the fully deoxygenated product from each multi-pass experiment, with a zoomed-in plot on the dimer region. Protocols and conditions described herein. (FIG. 4D) Production of fully deoxygenated lignin oil using optimized multi-pass conditions. The Sankey diagram shows the carbon balance on each pass of the optimum multi-pass experiment. The carbon content in the whole oil was determined using total carbon analysis. The monomer carbon content was determined with GC-FID analysis and is plotted as a fraction of the total carbon content. The carbon content was multiplied by the steady state recovery of the oil (by mass) across each pass. Photos show the oil at each stage of the process, with water (bottom phase) formed during the reaction separating from the deoxygenated organics (top phase). Conditions: 2.88 g Mo2C (60-100 mesh), 0.1 mL/min lignin oil, 90 mL/min H2, 900 psi, 350° C. first pass, 375° C. second pass, and toluene at 1 mL/min for 30 min during start-up.

FIG. 5 provides an example process for reductive catalytic fractionation as described herein utilizing poplar as a lignin source.

FIG. 6 provides an example process for hydrodeoxygenation and an example trickle bed reactor.

FIG. 7 illustrates example cycloalkane and aromatic products resulting from the described processes. The color or gray scale can corresponds to the graphs provided in FIGS. 8, 10A, 10B, 12A and 12B.

FIG. 8 provides feed compositions and monomer/oil ratios for polar, pine and corn stover feedstocks, illustrating a lower monomer to oil ratio for pine.

FIG. 9 provides GPC-UV chromatography for poplar, pine and corn stover feedstocks.

FIG. 10A shows monomer distribution for pine feedstock at different times for 350° C. and 400° C.

FIG. 10B shows the carbon, hydrogen and oxygen content for pine feedstock.

FIG. 11 illustrates the effect of feedstock ether content on fuel properties, indicating that 66% of products fall within jet fuel range aromatics.

FIG. 12 shows monomer distribution for corn stover feedstock at different times for 350° C. and 400° C.

FIG. 13 shows the elemental content for poplar, pine and corn stover feedstocks. Nitrogen and sulfur are likely due to proteins and extractives.

### DETAILED DESCRIPTION

The embodiments described herein should not necessarily be construed as limited to addressing any of the particular problems or deficiencies discussed herein. References in the specification to “one embodiment”, “an embodiment”, “an example embodiment”, “some embodiments”, etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described.

As used herein the term “substantially” is used to indicate that exact values are not necessarily attainable. By way of example, one of ordinary skill in the art will understand that in some chemical reactions 100% conversion of a reactant is possible, yet unlikely. Most of a reactant may be converted to a product and conversion of the reactant may asymptotically approach 100% conversion. So, although from a practical perspective 100% of the reactant is converted, from a technical perspective, a small and sometimes difficult to define amount remains. For this example of a chemical reactant, that amount may be relatively easily defined by the detection limits of the instrument used to test for it. However, in many cases, this amount may not be easily defined, hence the use of the term “substantially”. In some embodiments of the present invention, the term “substantially” is defined as approaching a specific numeric value or target to within 20%, 15%, 10%, 5%, or within 1% of the value or target. In further embodiments of the present invention, the term “substantially” is defined as approaching a specific numeric value or target to within 1%, 0.9%, 0.8%, 0.7%, 0.6%, 0.5%, 0.4%, 0.3%, 0.2%, or 0.1% of the value or target.

As used herein, the term “about” is used to indicate that exact values are not necessarily attainable. Therefore, the term “about” is used to indicate this uncertainty limit. In some embodiments of the present invention, the term “about” is used to indicate an uncertainty limit of less than or equal to ±20%, ±15%, ±10%, ±5%, or ±1% of a specific numeric value or target. In some embodiments of the present invention, the term “about” is used to indicate an uncertainty limit of less than or equal to ±1%, ±0.9%, ±0.8%, ±0.7%, ±0.6%, ±0.5%, ±0.4%, ±0.3%, ±0.2%, or ±0.1% of a specific numeric value or target.

As used herein, the term “Mo2C” or “Mo2C-based catalyst” refers to a catalyst comprising or consisting essentially of a form of molybdenum carbide. In some embodiments, the catalyst may comprise or consist essentially of Mo₂C or β-Mo₂C, but other forms or phases of molybdenum carbide may also be the primary or a secondary component. Further, Mo2C-based catalysts may include metal-doped catalyst where a metal, such as a transition metal, is included in the catalyst, for example, Cu, Ni, Pt. Mo2C based catalysts may also be multidimensional or multilayered.

The provided discussion and examples have been presented for purposes of illustration and description. The foregoing is not intended to limit the aspects, embodiments, or configurations to the form or forms disclosed herein. In the foregoing Detailed Description for example, various features of the aspects, embodiments, or configurations are grouped together in one or more embodiments, configurations, or aspects for the purpose of streamlining the disclosure. The features of the aspects, embodiments, or configurations, may be combined in alternate aspects, embodiments, or configurations other than those discussed above. This method of disclosure is not to be interpreted as reflecting an intention that the aspects, embodiments, or configurations require more features than are expressly recited in each claim. Rather, as the following claims reflect, inventive aspects lie in less than all features of a single foregoing disclosed embodiment, configuration, or aspect. While certain aspects of conventional technology have been discussed to facilitate disclosure of some embodiments of the present invention, the Applicants in no way disclaim these technical aspects, and it is contemplated that the claimed invention may encompass one or more of the conventional technical aspects discussed herein. Thus, the following claims are hereby incorporated into this Detailed Description, with each claim standing on its own as a separate aspect, embodiment, or configuration.

### Example 1—Continuous Hydrodeoxygenation of Lignin to Jet-Range Aromatic Hydrocarbons

Described in this example is a solvent-free process for the HDO of poplar RCF lignin oil with near-quantitative carbon yields using earth-abundant molybdenum carbide (Mo2C) catalysts. Continuous flow HDO experiments with real lignin feeds under solvent-free conditions were performed in a custom-built three-phase trickle bed reactor. Poplar RCF lignin oil was chosen as an illustrative lignin substrate, with recent studies showing potential for commercial implementation of RCF in a lignin-first biorefinery. The RCF process solvolytically extracts and catalytically depolymerizes lignin from whole biomass to produce an oxygenated aromatic oil consisting of ~50 wt % monomeric species and ~50 wt % carbon-carbon linked dimers and larger oligomers. Accordingly, complete deoxygenation of RCF oil would generate a mixture rich in alkylated arenes ranging between C9 and C20, which is ideal for jet fuel blending.

### Results

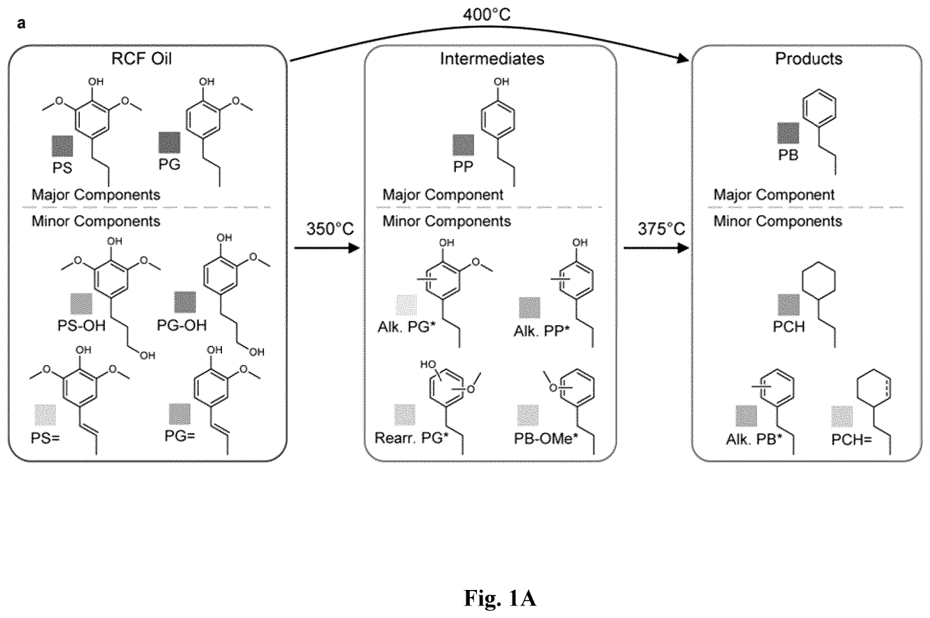

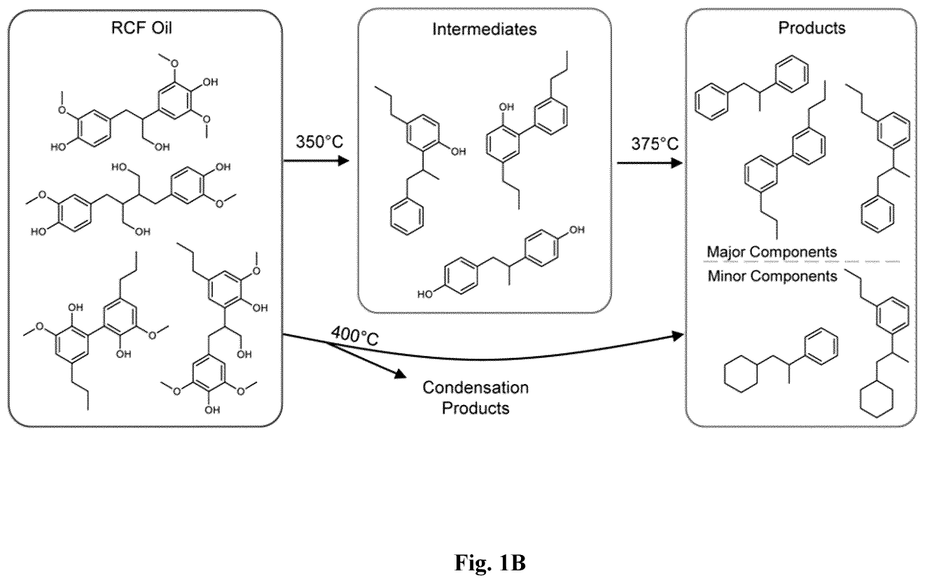

Tracking deoxygenation with Mo2C-based catalysts. The complexity of lignin oil components and their deoxygenated products necessitates rigorous analytical methods to close mass balances accurately. Accordingly, we identified and quantified partially and fully deoxygenated monomers and dimers during HDO experiments using a feed consisting of 2 wt % RCF oil dissolved in toluene Unsupported Mo2C was used as the catalyst due to its ability to directly cleave C—O bonds in model aromatic oxygenate molecules without hydrogenating the aromatic rings Although Mo2C has not been studied extensively for the deep deoxygenation of lignin oil, we hypothesized it could provide a route toward hydrogen-efficient deoxygenation with control over aromatic product selectivity. FIG. 1A summarizes all the lignin-derived monomers quantified by gas chromatography with flame ionization detection (GC-FID) in this study. This method afforded monomer mole balances of 100% +/−5% at all levels of partial conversion investigated. We used trimethylsilyl derivatization with GC coupled with mass spectrometry (GC-MS) to identify dimeric compounds at different levels of conversion (FIG. 1B). Generally, both monomeric and dimeric species followed similar conversion and selectivity trends to those observed during vapor-phase Mo2C HDO with model compounds. The first functional groups to undergo deoxygenation are the γ-hydroxyl groups (e.g., dihydroconiferyl alcohol, PG—OH), generating guaiacol and syringol derivatives. Next, deoxygenation of the methoxy group (e.g., propylguaiacol, PG) produces phenolics. Finally, the phenolic groups (e.g., propylphenol, PP) are cleaved to produce alkylbenzenes.

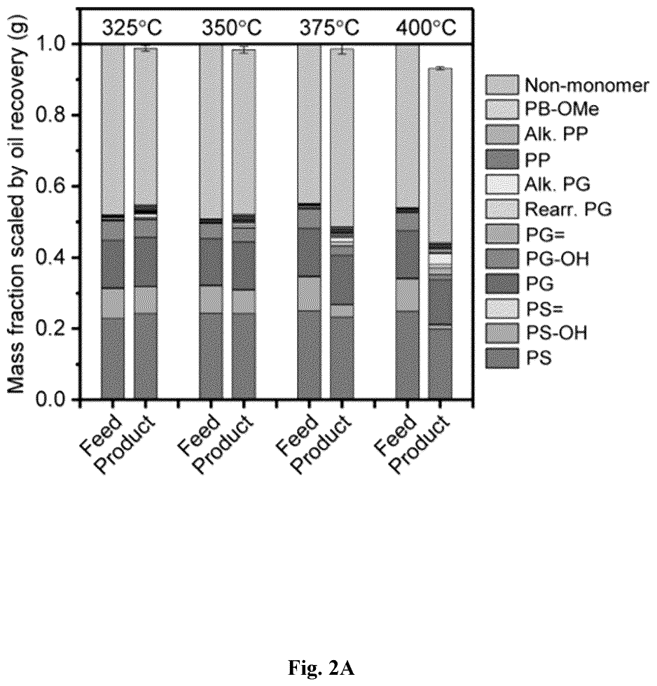

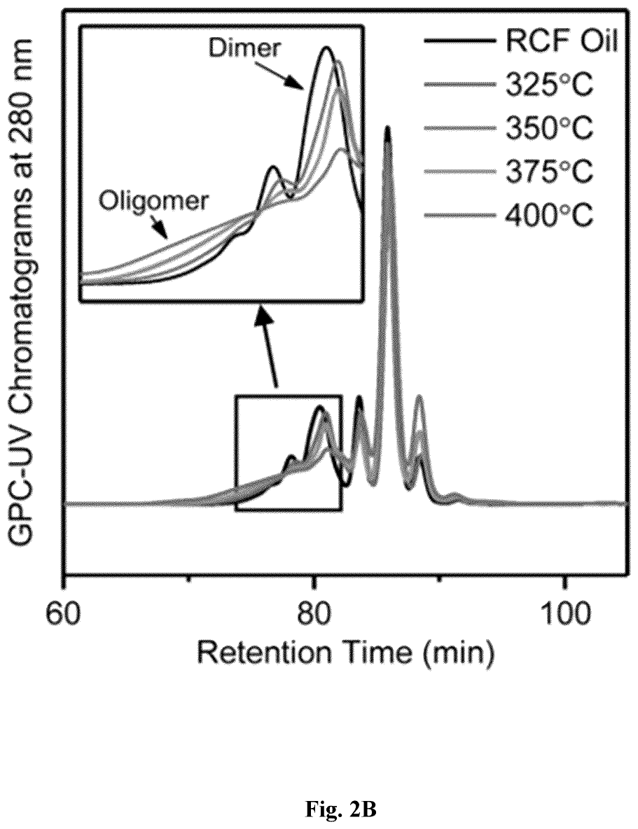

Evaluating solvent-free lignin oil stability. While 2 wt % RCF oil in toluene was a useful system for the development of analytical methods, toluene only dissolves ~70 wt % of the RCF oil, extracting low molecular weight components preferentially. For this reason, neat RCF oil was used as the feedstock for all subsequent studies. To study the stability of solvent-free RCF oil under elevated temperatures and determine acceptable operating temperatures for the HDO process, we performed catalyst-free flow experiments using conditions otherwise amenable to HDO chemistry. Reactor temperatures of 325, 350, and 375° C. showed lignin oil mass recoveries of 98.7 (±1.2), 98.5 (±2.0), and 98.6 (±2.5) wt %, respectively, whereas at 400° C., the mass recovery was 93.2 (±0.9) wt % (FIG. 2A). PG—OH and PS—OH, which make up 15 wt % of the RCF oil feed, were the most sensitive monomers to temperature, with combined recoveries of 90.0, 87.9, 40.2 and 17.8 wt % at 325° C., 350° C., 375° C. and 400° C., respectively. Additionally, a combined 5.6 wt % of rearranged, alkylated, and partially deoxygenated monomeric products were formed during the no-catalyst experiment at 400° C. Changes in the molecular weight distribution of the lignin oil, characterized using gel permeation chromatography (GPC) and GC-MS data of derivatized samples, indicated that increasing the temperature from 325 to 400° C. progressively decreased dimer concentrations and promoted the formation of new oligomers via condensation reactions (FIG. 2B).

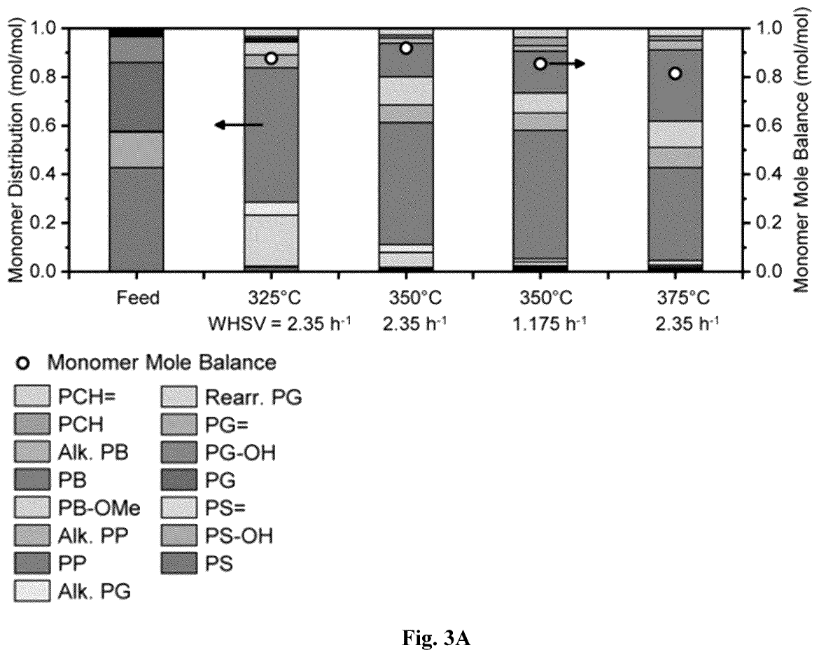

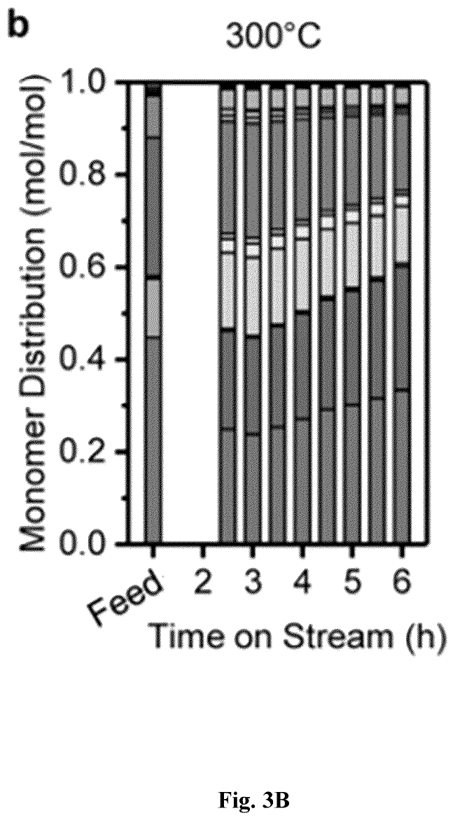

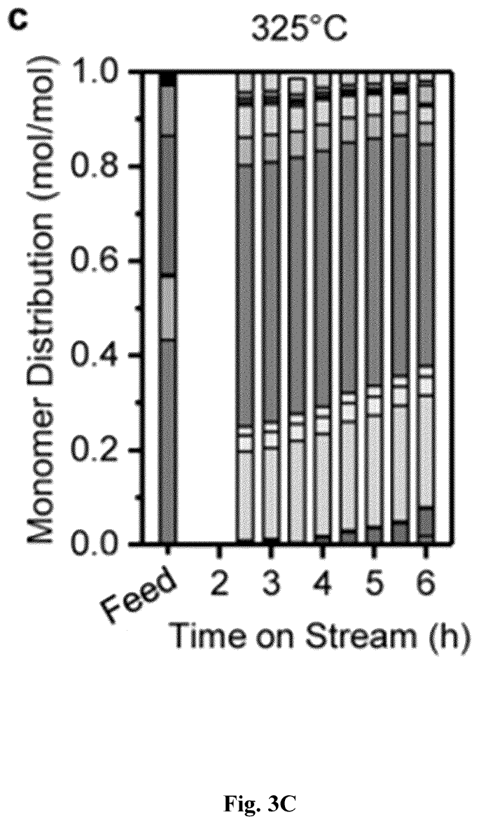

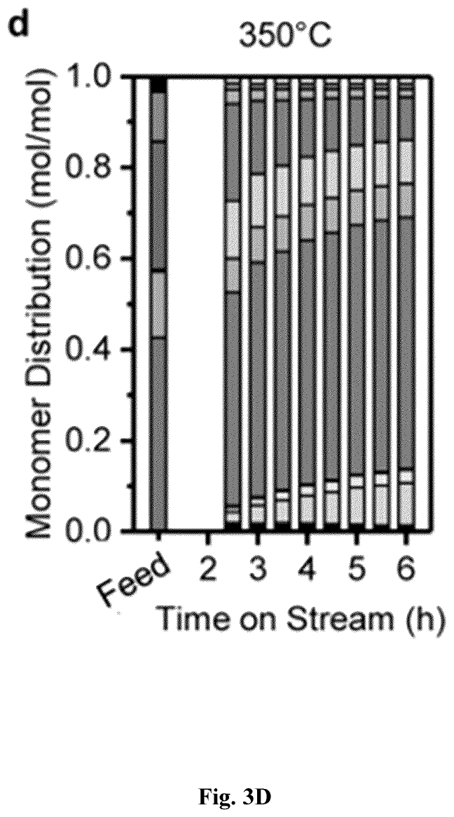

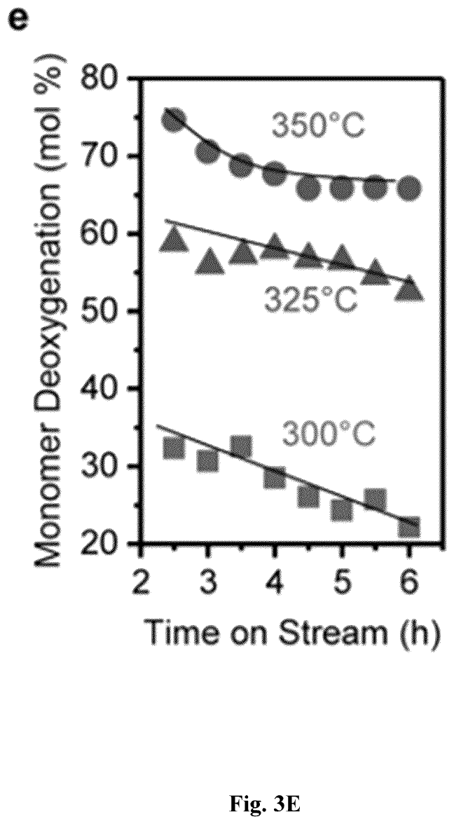

Mo2C catalyst activity during HDO of lignin oil. We next evaluated both the activity and stability of the Mo2C for the continuous HDO of undiluted lignin oil at temperatures below 400° C. to reduce deleterious lignin condensation (FIGS. 3A-3E). To avoid structural changes caused by the exposure of the carbide phase to molecular oxygen, fresh Mo2C was synthesized in situ via the carburization of ammonium molybdate tetrahydrate (AMT) immediately prior to the start of each experiment. At 350° C. and a weighted hourly space velocity (WHSV) of 2.35 g_{lignin}/g_{catalyst}/h, a steady state monomer oxygen conversion of 65.8+/−0.05 mol % was achieved with major monomeric products of 50.2 mol % PP, 13.8 mol % PB and 11.6 mol % PB-OMe (FIG. 3A). Despite achieving stable deoxygenation at these conditions, neither doubling the residence time (WHSV=1.175 h⁻¹), nor recycling the partially deoxygenated oil over a fresh catalyst bed at 350° C. resulted in complete monomer deoxygenation. Higher temperatures were required to activate the aryl-OH bonds, with 375° C. and a WHSV=2.35 h⁻¹ achieving 48.5% removal of phenolic groups. FIG. 3B and FIG. 3C show the effect of temperature on catalyst stability. Transient data collected at 300 and 325° C. from 2.5 to 6 h on stream showed steady deactivation profiles with a decrease in monomer oxygen conversion of 58 to 52 mol % for 325° C. and 32 to 22 mol % for 300° C. Although temperatures as low as 150° C. have been used to deoxygenate anisole and 280° C. to achieve near complete deoxygenation of model phenolic mixtures to aromatics at steady state with Mo2C, our data indicate that temperatures of 350° C. and 375° C. are needed for stable removal of methoxy and phenolic groups in neat lignin oil, respectively. These results on activity and stability resemble the performance of MoO₃ during HDO. Although PXRD analysis of the catalyst after reaction does not show bulk oxidation to MoO₃, in situ techniques have shown the surface of Mo2C is oxidized during reaction with oxygenates. This potentially explains why prior work on HDO of oxygen-rich real lignin feeds with Mo2C did not achieve complete deoxygenation and highlights the difficulty in translating the results of model systems to biomass feedstocks.

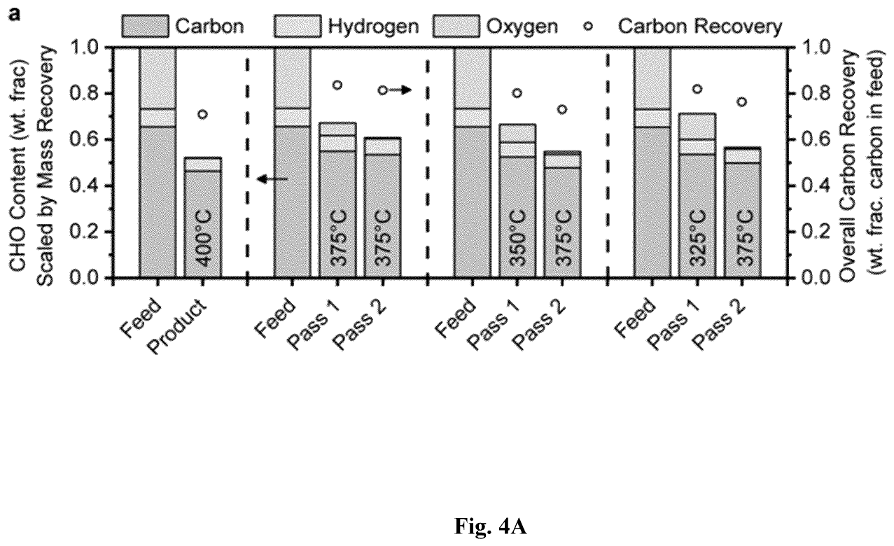

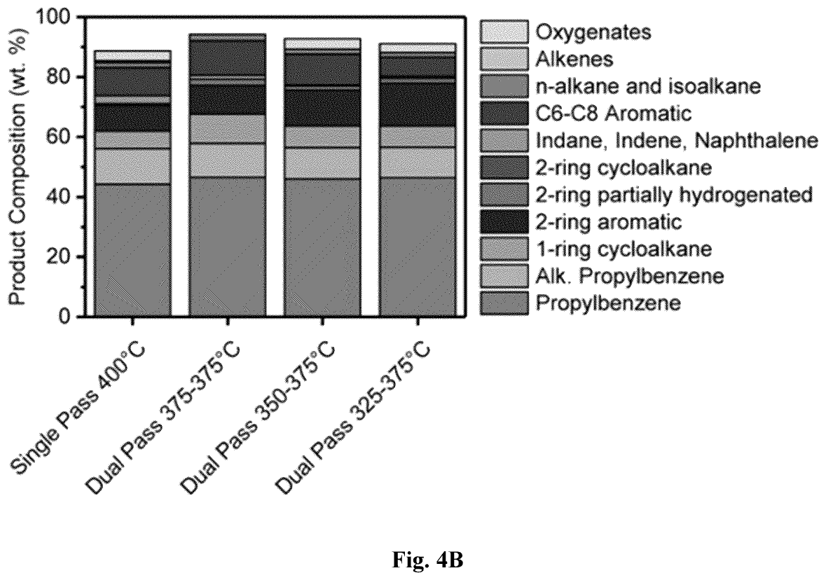

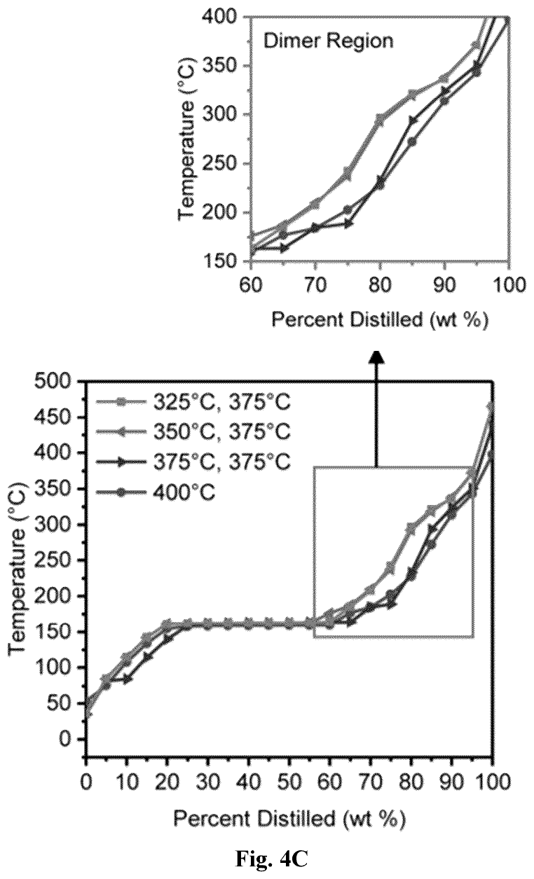

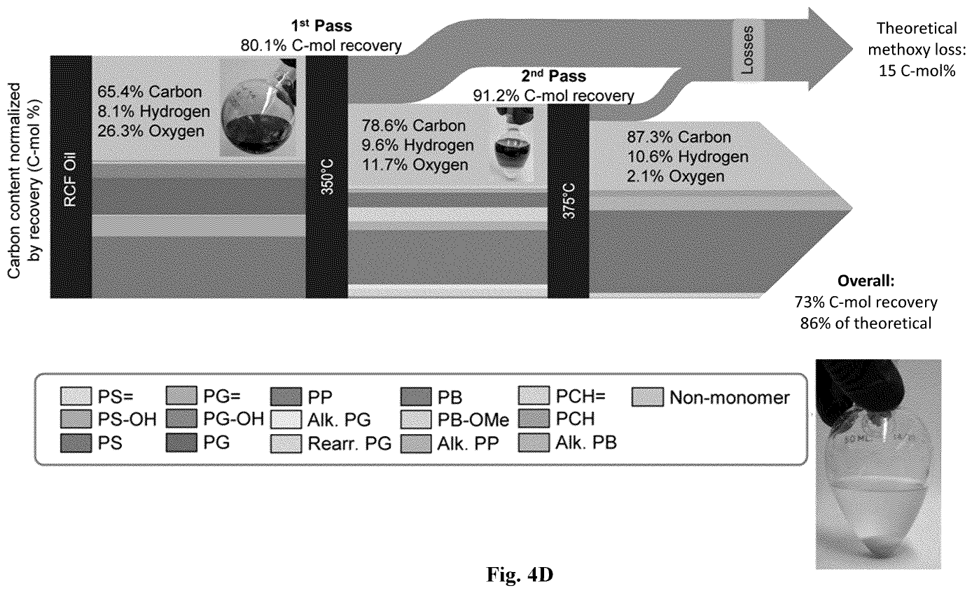

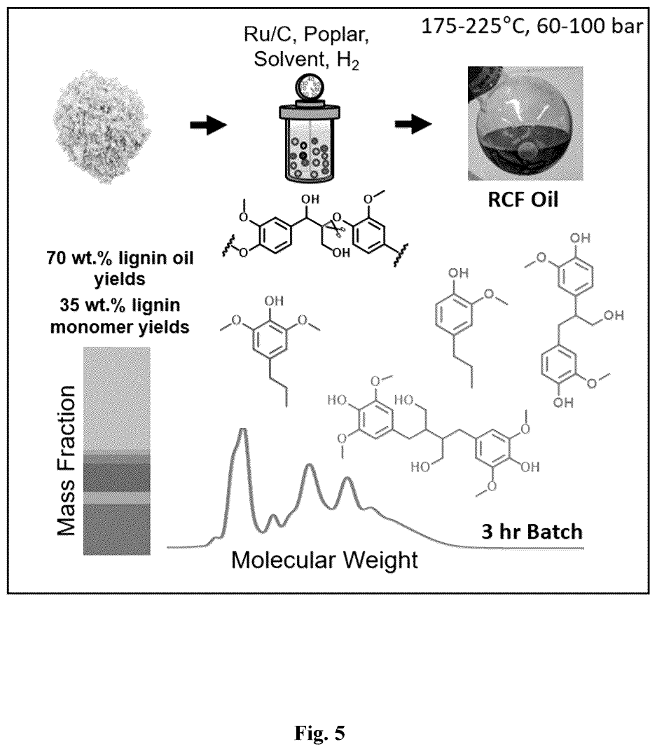

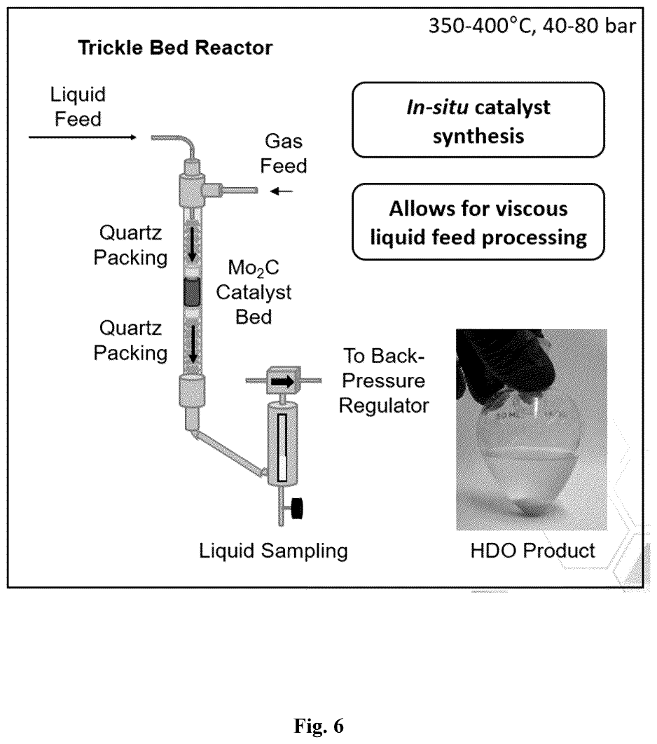

Achieving deep deoxygenation to aromatic hydrocarbons. Together, lignin oil stability (FIGS. 2A-2B) and catalyst activity (FIGS. 3A-3E) experiments revealed an important trade-off in lignin oil HDO: low temperatures result in catalyst deactivation but achieve high carbon balances by preserving oligomers, while high temperatures are necessary to maintain catalyst activity and achieve full deoxygenation, but result in the loss of dimers through condensation. We evaluated two routes for generating deeply deoxygenated oil products: i) increasing the temperature and the residence time in a single-pass at 400° C. and WHSV=1.175 h⁻¹ and ii) operating in a two-pass mode wherein an initial HDO run is performed at an intermediate temperature to generate a mixture of partially deoxygenated monomers and oligomers that is more resistant towards subsequent condensation reactions, followed by a second pass at a higher temperature to achieve full deoxygenation. For the two-pass experiments, first-pass products were collected at 325° C., 350° C., or 375° C., each over a fresh catalyst bed. Next, these products were deoxygenated at 375° C. in a second run with a fresh catalyst bed. FIG. 4A summarizes the carbon, hydrogen, and oxygen content of each oil scaled by the mass recovery from each pass along with the overall carbon recovery. After the first pass at 400° C., 375° C., 350° C., and 325° C., 0.7, 8.0, 11.7, 15.8 wt % oxygen remained, respectively. This was reduced to 1.0, 2.1 and 1.2 wt % for 375-375° C., 350-375° C., and 325-375° C., respectively. The near complete deoxygenation of the products was confirmed with heteronuclear single quantum coherence (HSQC) nuclear magnetic resonance spectroscopy (NMR) and derivatization+GC-MS. The final monomer product distributions were similar in all cases, with propylbenzene selectivity values of ca. 78 mol %. The dimer products were more sensitive to temperature, showing 8.9, 9.5, 11.9, and 14.2 wt % dimer content for first pass temperatures of 400° C., 375° C., 350° C., and 325° C., respectively as determined by quantitative GCxGC analysis (FIG. 4B). This trend in dimer recovery was supported by simulated distillation (FIG. 4C), and additional chromatography techniques.

Two-pass operation with 350° C. followed by 375° C. achieved an optimal balance for catalyst stability and jet-range aromatic production. Indeed, 350° C. was the optimal temperature to maintain stable catalyst activity at partial deoxygenation in the first pass (total product oxygen content of 11.7%), while higher temperatures of 375° C. and 400° C. resulted in an elevated loss of aromatic dimers. FIG. 4D summarizes the optimal overall process, where we obtained a reduction in total oxygen content from 26.3 mol % to 2.1 mol % (95.7% conversion), with an unprecedented 73.1 C-mol % recovery (86% of theoretical based on removal of methoxy groups) and 87.5% monomer selectivity toward aromatic hydrocarbons. For reference, recent reports have shown yields between 10-30 wt % and 30-50 wt % via one-and two-step processes, respectively, while our process's mass recovery is consistently between 50-60 wt % while achieving higher levels of deoxygenation than those studies discussed. GCxGC quantification determined a final aromatic dimer (C14-C20) content of 11.9 wt. % and aromatic monomer (C9-C12) content of 56 wt %, summing to 67.9 wt % of jet-range aromatics in the final oil.

### Discussion, Recommendations and Conclusions

The provided example demonstrates that lignin stability is a key constraint under typical HDO conditions, and that two-pass HDO, with the first pass used for stabilization via partial conversion and the second pass for deep deoxygenation, is a viable strategy for lignin upgrading with high atom economy. This strategy could be broadly applicable to other lignin feedstocks. Furthermore, HDO reactions with Mo2C selectively cleaved C—O bonds while leaving the C—C structure of the lignin substrate intact. In addition to minimizing the consumption of hydrogen, this selectivity indicates that the distribution of HDO products could be tuned by modifying the lignin feedstock itself. For example, a lignin substrate with a higher fraction of dimeric components could be utilized to increase the C14-C20 aromatic fraction in the resulting deoxygenated product. Furthermore, the carbon backbone of the dimer and oligomer fraction retains sufficient chemical functionality during reaction to differentiate between types of lignin inter-unit linkages that are synthesized in planta (e.g., β-1 vs β-5), providing an analytical opportunity to quantitatively track lignin bond distributions.

This example motivates the development of catalysts that can achieve stable HDO of real lignin feedstocks at low temperatures. Notably, the behavior of the catalyst under reaction with neat lignin oil was distinctly different than vapor-phase model compound studies reported to date. Furthermore, while the existing literature contains a strong fundamental framework from which to understand the impact of controlled Mo2C surface oxidation on model compound reactivity, the parameters that determine extent of catalyst oxidation under reaction conditions with real lignin feeds have not been mapped and are not well understood. Understanding the factors that drive catalyst surface deactivation under reaction conditions with real feedstocks may enable rational catalyst design or reaction engineering solutions to mitigate catalyst deactivation and enable lower operating temperatures. Altogether, performing experiments in a trickle-bed reactor to generate continuous, steady-state HDO data using a real lignin feedstock, along with rigorous analytics to track carbon balances and individual components, was essential to understanding the limitations of this complex system.

Deoxygenated aromatics resulting from this process could be directly blended with existing commercial SAFs (which mainly consist of iso and n-alkanes), potentially overcoming the existing 50% blend-wall by providing the necessary aromatic components to increase both fuel density and elastomeric compatibility. Alternatively, subsequent hydrogenation, ring-opening, and/or hydrocracking reactions could be implemented to convert a fraction of the lignin-derived aromatics to other paraffinic compounds (including naphthenes and iso/n-alkanes), thereby generating an entirely lignin-derived SAF.

Finally, we propose potentially blending feedstocks (rather than deoxygenated products) to achieve desired aromatic/aliphatic ratios followed by subsequent deoxygenation over Mo2C to directly generate a complete drop-in SAF. Ongoing techno-economic analyses and life cycle assessments are geared towards identifying critical areas for further development and integration of our product into a 100% SAF blend.

### Materials and Methods

Generation of RCF lignin oil. 60 g of poplar (milled and sieved to <2 mm), 12 g of 5 wt % ruthenium on carbon (Sigma-Aldrich®) and 400 mL methanol (ACS reagent grade, Macron Fine Chemicals) were loaded into a 1 L batch reactor (Parr instrument company®, Series 4525HP). The reactor was sealed, flushed 3 times and pressurized to 30 bar with H2 (UHP, Airgas®). During reaction, the mixture was stirred at 700 rpm with an overhead impeller. The temperature was monitored using an internal thermocouple and the furnace, stir rate, and cooling water were controlled with a Parr® instrument controller (series 4848). The temperature was ramped from 25° C. to 225° C. for 1.5 h, then held at 225° C. for 3 h before rapidly cooling to 25° C. via cooling water. Catalyst loading, hydrogen pressure, and reaction time were optimized to achieve complete conversion of ether bonds, ensuring no additional monomers were produced during HDO to enable monomer mole closure.

After reaction, the lignin fraction was isolated by vacuum filtration through a 4 μm ceramic filter. The solids were rinsed twice with methanol and the solvent was removed under vacuum. The resulting oil was purified to remove any sugars or acids that were extracted from the biomass during RCF with a dichloromethane (DCM)/water extraction, starting with 30 mL DCM (Laboratory-plus grade, Honeywell®) and 30 mL water (Milli-Q®) and subsequently rinsing the aqueous phase twice with 15 mL DCM. The DCM was removed from the organic phase under vacuum, with a preliminary removal at 100 torr by rotary evaporation followed by a secondary 0.1 mTorr evacuation on a Schlenk line with magnetic stirring at 250 rpm. Overall, this process obtained ~9 g of purified RCF lignin oil from a single 60 g poplar RCF experiment.

Catalyst synthesis. AMT (ACS reagent grade, Sigma-Aldrich®) was sieved between 60-100 mesh and an appropriate amount to achieve the desired quantity of Mo2C was loaded into the trickle-bed reactor (see loading procedure). The reactor was then heated from 25° C. to 700° C. over 3.5 h and held at 700° C. for 3 h. Hydrogen gas was flowed at 55 mL/min for the duration of the synthesis, and methane (UHP, Airgas) was flowed at 15 mL/min for the first 6 h, enabling a 0.5 h scavenging step with pure hydrogen gas at 700° C. The reactor was then cooled and left sealed under hydrogen gas flow prior to reaction.

Trickle bed reactor design. Reactions were performed in a custom trickle-bed reactor. A 21″ long, ¼″ OD Hastelloy reactor tube was heated in a vertically-mounted, insulated, single-zone, split furnace (Applied Test Systems® Series 3210) with steel blocks (machined to fill void space in the furnace) to ensure adequate heat transfer and maintain isothermal operation. A K-type thermocouple slotted to contact the outside of the reactor tube was used to measure temperature while another thermocouple at the middle of the furnace and contacting the outside of the steel blocks was used to regulate temperature with a PID temperature controller (Digi-sense® TC9500). Gas flow rates were controlled using mass flow controllers (Brooks® SLA5850S1BAB1B2A1) and liquid was pumped using a Teledyne® ISCO syringe pump (Model 500D). Liquid was fed into the top of the reactor through a 1/16″ OD 316 stainless steel tube which extended to the start of the heating zone. Gas was fed into the top of the reactor via ¼″ 316 stainless steel tubing and flowed co-currently with liquid in the down-flow direction through the packed catalyst bed. Liquid samples were collected at room temperature in a gas/liquid separator (Jerguson Gage & Valve Co.®). Gas flowed out of the top of the gas/liquid separator and through a diaphragm back-pressure regulator (Equilibar® H3P1SNN8-NSBP1500T100G20KK), which maintained the overall system pressure. A nitrogen back-fill line enabled system pressure maintenance during sampling through a needle valve (Swagelok®).

Reactor packing procedure. A quartz wool (Technical Glass Products Inc.®) plug was placed in the bottom of the reactor tube, followed by 9.75″ of quartz chips (fused silicon dioxide (granular), Sigma-Aldrich®), filling from the bottom of the reactor to the center of the heating zone of the furnace. A quartz wool plug, followed by the catalyst bed, followed by another quartz wool plug were then loaded. For neat lignin oil experiments, the bed comprised pure catalyst only. For 2 wt % lignin oil in toluene experiments, the catalyst was diluted to 1 g using 120 grit silicon carbide powder (≥98.0%, Alfa Aesar®). Finally, quartz chips were added up to 1″ below the level of the drip tube at the top of the reactor.

Feed preparation. Neat lignin oil was used directly after the procedure outlined above for generating RCF oil, combining the oil generated from 5-10 batch reactions to generate 45-90 g of feed oil. Recycled lignin oil was prepared by combining the samples from a previous experiment(s) after removing enough sample for analysis. The water fraction phase-separated from the partially deoxygenated lignin oil and was removed with a pipette. Diluted 2 wt % lignin oil in toluene was prepared by adding 2 wt % lignin oil, 0.2 wt % decane (≥99.0%, Sigma-Aldrich®), and 97.8 wt % toluene (≥99.8, VWR Chemicals® BDH) to a large glass container. It was then shaken vigorously, sonicated for 20 minutes, and left overnight to equilibrate. To remove the suspended undissolved lignin oil and prevent clogging, the cloudy lignin oil in toluene was centrifuged at 8,000 rpm for 5 minutes. The resulting liquid was decanted and used for reaction.

Running a reaction. After catalyst synthesis, the reactor was cooled to room temperature at atmospheric pressure under flowing hydrogen at 55 mL/min. The steel heat transfer blocks were placed in the furnace and the reactor was slowly pressurized by nitrogen back-fill to the reaction pressure of 900 psi. The hydrogen flowrate was set to 30 mL/min for reagents dissolved in toluene or 90 mL/min for neat lignin oil. The reactor was leak checked and heated. For reagents dissolved in toluene, the feed was flowed at 2 mL/min during heating to pre-wet the catalyst bed, and the flowrate was reduced to the reaction flowrate once the furnace reached temperature. The liquid collector was drained when the furnace reached 200° C., and samples were taken when the furnace reached 250° C. and 300° C. Samples were taken every 40 min for the duration of the experiment. For neat lignin oil experiments, the reactor was heated to reaction temperature while flowing pure toluene at 1 mL/min. Once the reactor reached temperature, the liquid feed was switched to neat lignin oil flowing at 0.1 or 0.05 mL/min. Samples were taken every 30 or 60 min.

Sample work-up and analysis. Each sample was collected into a pre-weighed vial, allowing for sample mass measurement post-experiment. To prepare neat lignin oil samples for gas chromatography with flame ionization detection (GC-FID) analysis, 30 μL of oil was dissolved in 2 mL of acetone containing 2 mg/mL of 1,3,5-tri-tert-butylbenzene (≥98.0%, TCI®), measuring the weight of oil and acetone solution to improve accuracy. Samples from experiments with reagent dissolved in toluene were weighed and injected on GC-FID with no additional work-up procedure, using the contained decane internal standard. To prepare samples for NMR analysis, 100 mg of oil was dissolved in 500 μL acetone-d6 (≥99 atom % D, Acros Organics®).

Quantification of monomers with GC-FID. An Agilent Technologies® 7693 autosampler injected a 1 μL volume of each prepared sample into an Agilent® 7890A GC system. The GC method utilized a split ratio of 10:1, an inlet temperature of 280° C. and a ramp rate of 10° C./min from 50° C. to 280° C. followed by a 10-minute hold at 280° C. for a total run time of 29 min. The GC was outfitted with a 30 m×250 μm×0.25 μm Agilent Technologies® HP-5MS column and a flame ionization detector (FID) was used to quantify the products.

The GC-FID was calibrated with methylcyclohexane (≥99.0%, TCI)®, toluene (≥99.8%, VWR Chemicals® BDH), propylcyclohexane (≥98.0%, TCI), propylbenzene (≥99.0%, Sigma-Aldrich®), 4-propyltoluene (≥99.0%, TCI®), p-propylanisole (≥99.0%, Sigma-Aldrich®), 4-propylphenol (≥99.0%, Sigma-Aldrich®), 2-methoxy-4-propylphenol (propylguaiacol) (≥99.0%, Sigma-Aldrich®), isoeugenol (≥98.0%, Sigma-Aldrich®), 4-allyl-2, 6-dimethoxyphenol (≥95.0%, Sigma-Aldrich®) and dihydroconiferyl alcohol (synthesized in-house). Relative response factors (RFs) were generated for each of these compounds relative to decane (for experiments performed in toluene with decane as an internal standard) and 1,3,5-tri-tert-butylbenzene (for neat lignin oil experiments). Additionally, decane was calibrated with a relative response factor to 1,3,5-tri-tert-butylbenzene to enable accurate quantification of decane in the lignin oil in toluene feeds. RFs were calculated by fitting the line of relative mass fraction vs area ratio of the compound of interest to the standard. Adjustment factors were used to generate approximate RFs for products that were not available commercially. For each sample, the peak areas corresponding to known products were integrated. The ratio of compound peak area and standard peak area was calculated and multiplied by the RF and mass fraction of standard to obtain the mass fraction of compound. The mass fractions were then used for subsequent calculations of mole balance, conversion, yield, etc.

Gas chromatography and mass spectrometry (GC-MS). Analysis was performed using an Agilent Technologies® 7820A GC System outfitted with an HP-5MS Ultra Inert 30 m×250 μm×0.25 μm column and an Agilent® 5977B single quadrupole MS detector. Monomeric compounds in the feed and product were identified by injecting identical samples on a GC-FID and a GC-MS using the same column and method. The products were identified using the fragmentation patterns in GC-MS and matched with the same retention time observed in GC-FID.

Dimers were analyzed using derivatization followed by GC-MS, similar to our previously published method. Solvent-free lignin oil samples were dissolved in tetrahydrofuran (HPLC Grade, VWR Chemicals® BDH) (THF) at a concentration of 10 mg/mL. The derivatization reaction was performed by combining 600μL of 10 mg/mL lignin oil solution, 50 μL of pyridine and 100 μL of silylating agent [N,O-Bis(trimethylsilyl)-trifluoroacetamide (BSTFA) with 1% trimethylchlorosilane (TMS) (Sigma-Aldrich®)], then heating for 20 min at 50° C. BSTFA reacts readily with water, so it was ordered in 1 mL ampules and used immediately after opening.

For analysis, 1 μL samples were manually injected into the GC-MS. The method utilized a split ratio of 10:1, a split flow of 12 mL/min, and an inlet temperature of 280° C. The oven was programmed to ramp from 150° C. to 300° C. at a rate of 5° C./min and held at 300° C. for 18 min for a total run time of 49 min. GC-MS spectra were analyzed by comparing with published dimer structures and by predicting structures that may be present and comparing them with unknown MS spectra.

Gel permeation chromatography (GPC). Gel permeation chromatography was performed following a procedure similar to our previously published methods. Solvent-free oil samples were prepared for gel permeation chromatography by dissolution in THF at a concentration of 2 mg/mL (HPLC Grade, VWR Chemicals® BDH) and filtration using a 0.2 μm PTFE syringe filter. 20 μL of each sample was injected using a Hewlett Packard 1100 series autosampler. THF was used as the carrier solvent at a flowrate of 0.3 mL/min. Three 5 μm PLgel Agilent GPC columns (10⁴ Å, 10³ Å, and 50 Å) were arranged in series in order of decreasing pore size. The columns were maintained at a constant temperature of 26° C., and the system ran at a pressure of approximately 30 bar (a function of the flowrate, column choice, and temperature). A UV diode array detector at a wavelength of 280 nm with a reference wavelength of 360 nm and a 4 nm slit was used to analyze eluents.

NMR spectroscopy. NMR spectra were acquired on a Bruker® Advance Neo 400 MHz equipped with a 5 mm broad band observe (BBFO) SmartProbe, and spectral processing used Bruker's TopSpin® 4.0.8 (Windows) software. The central solvent peaks (acetone-d6) were used as the internal references (δC/δH: 29.84/2.05 ppm). Standard Bruker® implementations of the traditional suite of 1D and 2D [gradient-selected and ¹H-detected; for example, ¹H-¹³C HSQC] NMR experiments were used for structural elucidation and assignment authentication for monomers and oligomers. Processing used typical matched Gaussian apodization in F2 (LB=−0.1; GB=0.001) and squared cosine-bell apodization in F1.

Quantitative GCxGC TOF-FID Analysis. Detailed characterization of deoxygenated hydrocarbon products was conducted by comprehensive two-dimensional gas chromatography with simultaneous time-of-flight mass spectrometry and flame ionization detection (GCxGC TOF-FID). Samples were prepared for analysis by dilution (1:10 gravimetrically in acetone). Analysis was conducted using a LECO Pegasus® 4D system (LECO Corp®) equipped with a liquid nitrogen cooled thermal modulator and post column flow splitter. The primary column was a Rtx-17 sil, 20 m×180 μm×0.18 μm and the secondary column was a ZB-5HT, 1.0 m×180 μm×0.18 μm. The method utilized a 1.0 μL injection, split ratio 100:1, and injector temperature of 300° C. The primary oven held at 35° C. for 5 minutes prior to 3° C./min ramp to 125° C., 10° C./min ramp to 350° C., and hold for 1 min. The secondary oven was 40° C. offset from primary and the modulator was 15° C. offset from secondary. The modulator period was set to 8 seconds, with 1 s hot pulse and 3 s cold pulse from the start of run to 800 s, followed by 2 s hot pulse and 2 s cold pulse to end of the run. The transfer line to the mass spectrometer and FID were held at 350° C. The TOF mass range was m/z 29-600, with an acquisition rate of 200 spectra/s, and solvent delay of 70 s. The FID flows were set at 40 mL/min H2, Air flow of 300 mL/min and N2 flow of 25 mL/min. Compounds were identified using library matching and utilization of retention time regions in 2D chromatograms using LECO ChromaTof® Software. The FID signal was calibrated using a series of representative compounds to establish linearity and accuracy. All linear calibrations resulted in R²≥0.995. Compounds were quantified from their theoretical response factors calculated via effective carbon numbers.

Qualitative GCxGC FID/MS analysis. Qualitative characterization of deoxygenated products was done using two-dimensional gas chromatography with coupled flame ionization detection and mass spectrometry (GCxGC FID/MS). This analysis was conducted using an Agilent® 7890A GC with an Agilent 5975C Inert XL MSD. The system was equipped with a chiller-cooled (PolyScience® P10N4A101B) thermal modulator (Zoex ZX10711). Samples were injected neat post-reaction. Data analysis was completed using the software package Canvas™ v4.

The primary column used was a Rxi-5HT, 30 m×0.25 mm×0.25 μm while the secondary column used was a BPX50, 2 m×0.15 mm×0.25 μm. The method utilized a split ratio of 50:1, a 5 μL injection, and an injector temperature of 350° C. The primary oven was held at 45° C. for 1 minute followed by a ramp to 300° C. at 3° C./min. This temperature was then held for 5 minutes. The secondary column was offset from the primary by 25° C. while the thermal modulator thermal modulator was offset from the secondary by 5° C. The modulator used a 16 second period with a 375 ms hot jet duration. The transfer line to the MS was held at 250° C. The mass range was set to 40-550 m/z, with an acquisition rate of 50 spectra/s and no solvent delay. The FID detector was held at 300° C. with an H₂ flow of 30 mL/min, an air flow of 400 mL/min.

Total carbon, hydrogen, and oxygen (CHO) analysis method. Total carbon and hydrogen were determined by combustion using a LECO® Series CHN628 elemental analyzer (LECO® Corp). Oxygen content was calculated by difference.

Simulated distillation. Due to limited sample volume, distillation temperatures were determined by simulated distillation rather than physical distillation (i.e., ASTM D86). Boiling range distributions of deoxygenated oils were measured by simulated distillation following ASTM method D2887-19. An Agilent® 7890A GC equipped with cool-on-column inlet and FID was used. Method settings were used as found in ASTM D2887 Table 1 for an open tubular column (option 7). The column used was an MXT-1HT, 10 m×530 μm×2.65 μm (Restek Corp®). Samples were diluted 1:10 by volume in carbon disulfide for analysis. An injection volume of 0.1 μL was injected onto the analytical column. Boiling point calibrations were made using prepared standards purchased from Sigma-Aldrich® (Part #500658) and accuracy was verified with standard reference gasoil as described in D2887. Data analysis was conducted using Separation Systems SimDis Expert® 9.

Mass balance on neat lignin HDO experiment. To calculate a mass balance, a single-pass neat lignin oil experiment was performed in which a steady-state sample was accumulated for 3 hours. The ~18 mL sample was collected into a pre-weighed flask. The total sample was weighed, then a pipette was used to remove the aqueous phase. The remaining organic phase was weighed and characterized. The mass flowrate of the lignin oil feed was measured in triplicate by weighing the oil collected for 4 minutes at the experimental flowrate directly at the outlet of the ISCO pump. The mass flowrate was multiplied by the steady-state collection time to obtain the feed mass. The feed mass and steady-state oil mass were multiplied by the carbon mass percent of each sample to obtain the carbon mass recovery.

Solids compositional analysis procedure. Compositional analysis of the poplar samples was performed based on NREL's published guidelines. The poplar used was a reference material from Idaho National Laboratory, (Source: Morrow County, Oregon, Harvested: 2013, Hybrid Clone: Populus deltoides x Populus nigra, clone OP-367). Samples were first extracted with a high pressure and temperature flow through aqueous rinse followed by an ethanol rinse. The biomass was then treated with 72 wt % sulfuric acid for 1 h at 30° C. with mixing. The slurry was then diluted with water to 4 wt % sulfuric acid and heated in an autoclave for 1 h at 121° C. Klason lignin content was determined gravimetrically by filtering the acid insoluble residue out of the acidified aqueous slurry. The Klason lignin was combusted to determine the ash content in the biomass. The acid soluble lignin was quantified by UV/Vis spectroscopy (Thermo Scientific Nanodrop® 8000 spectrophotometer). Lignin absorbance measurements were performed at 240 nm and quantified with an extinction coefficient of 2.5. The sugar content was determined by high performance liquid chromatography (HPLC, Agilent® 1100 HPLC) using a refractive index detector (RI) kept at 55° C. A Shodex® Sugar SP0810 column equipped with a guard column was used for analysis at 85° C. with a flow rate of 0.6 mL/min of HPLC grade water as the mobile phase.

Catalyst characterization with powder X-ray diffraction. Powder X-ray diffraction (PXRD) was performed on a Bruker® D8 diffractometer using Cu Kα radiation, a step size of 0.02°, and a step time of 0.2 s.

### Example 2—Comparison of Poplar, Pine and Corn Stover Feedstocks

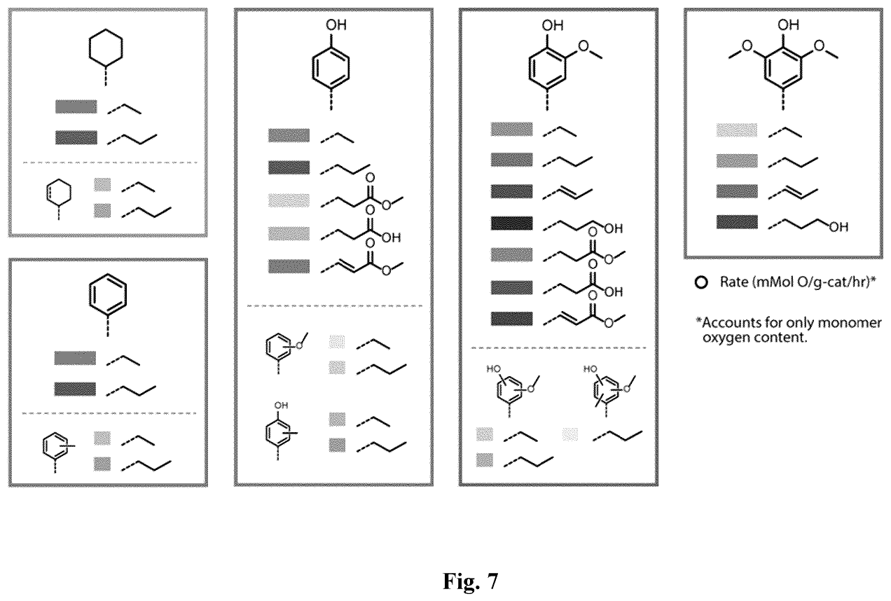

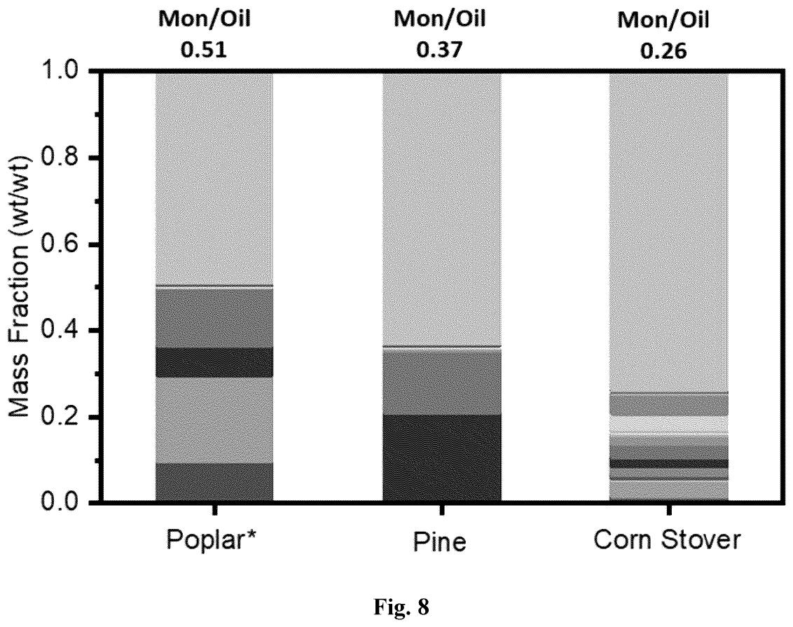

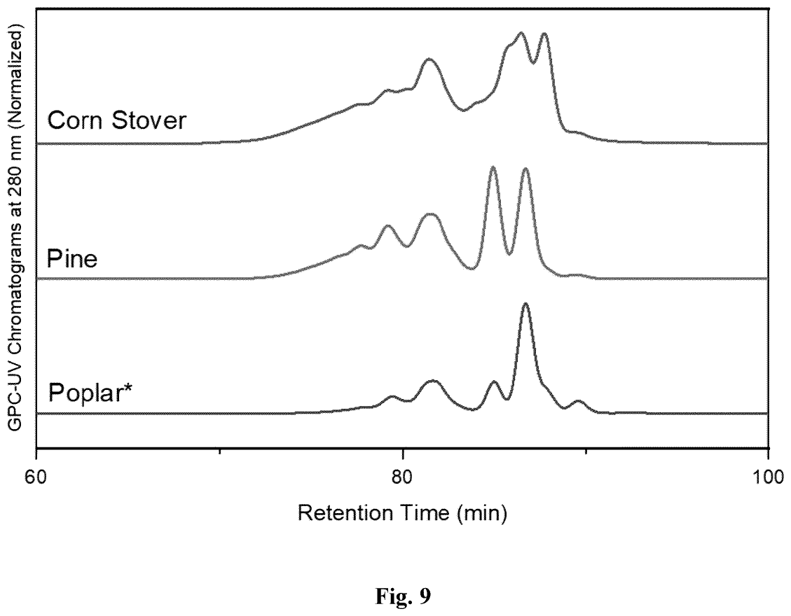

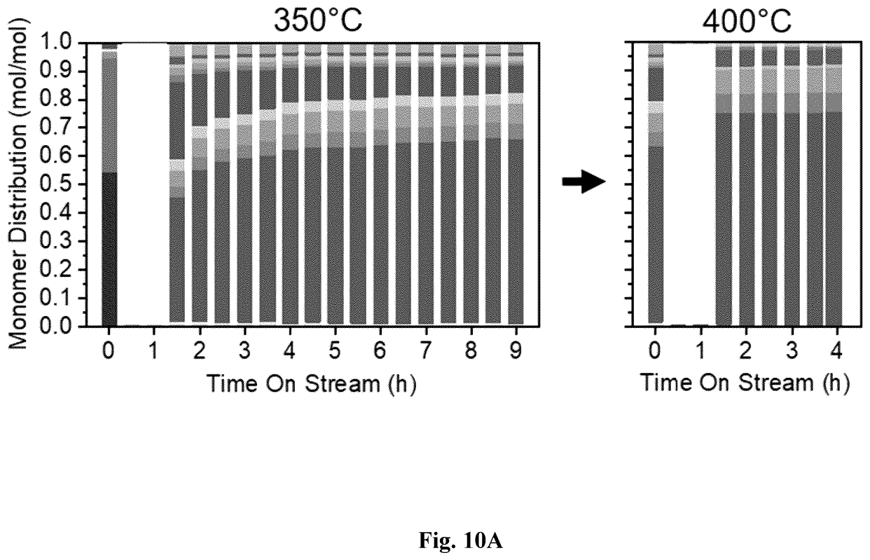

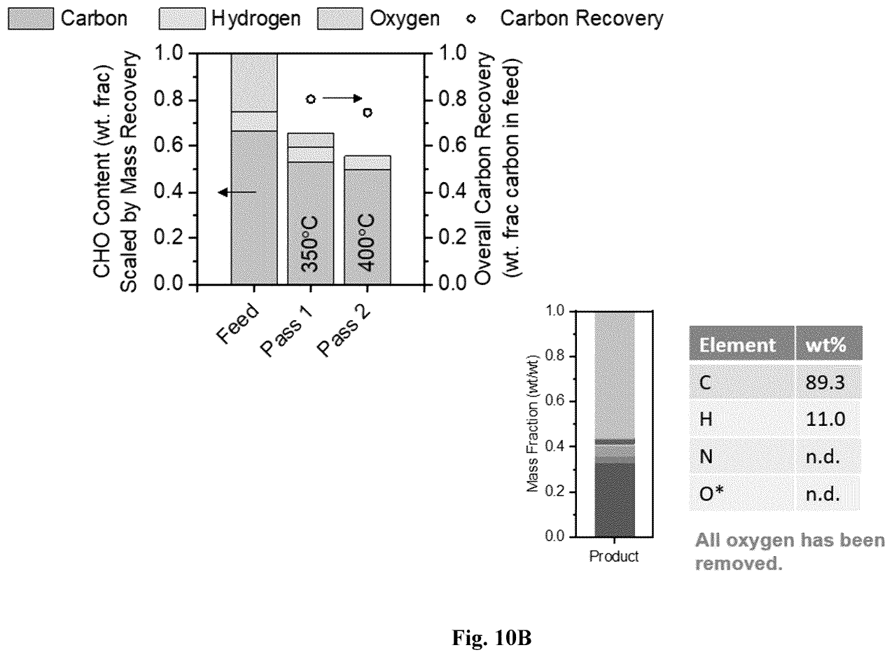

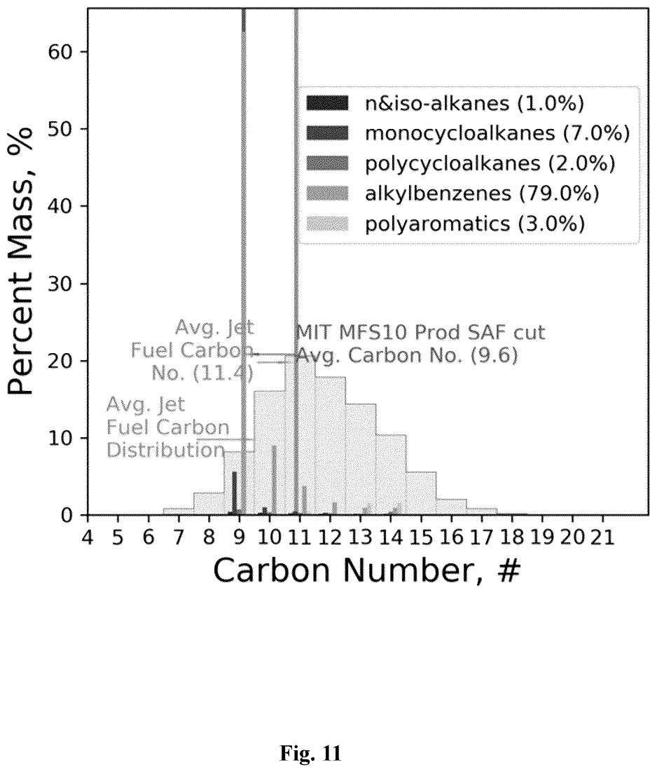

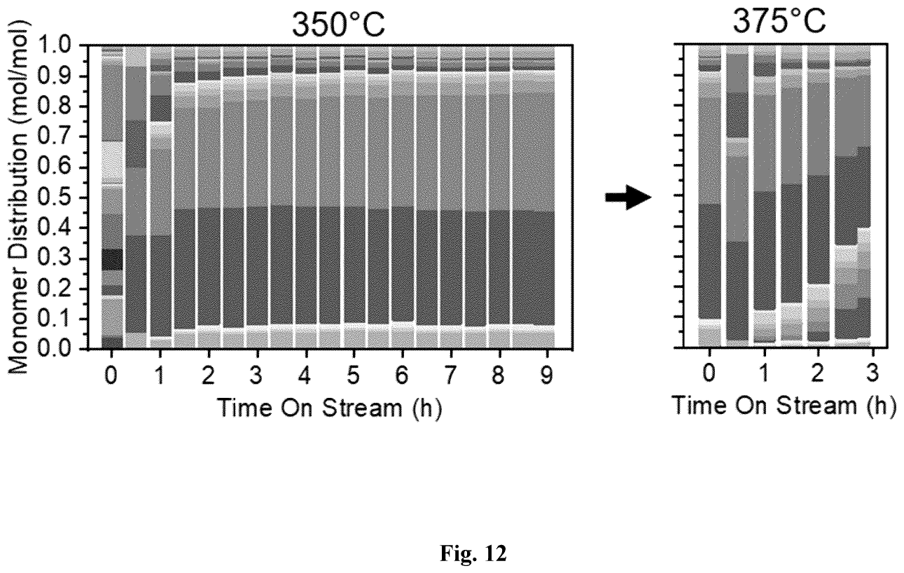

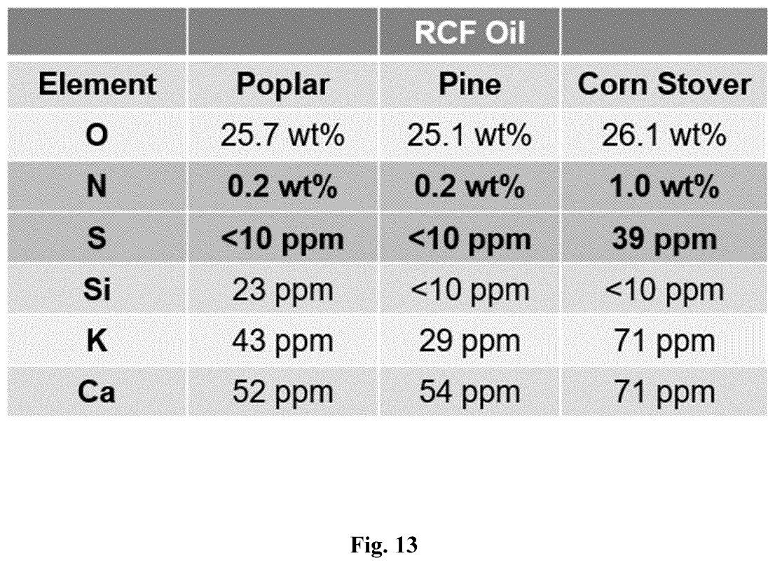

Comparison of various lignin feedstocks is provided in FIGS. 8-12. FIG. 7 provides example monomers corresponding to jet-range cycloalkanes and aromatics at can be generated by the methods provided herein.

The described invention can be further understood by the following non-limiting examples:
Example 1. A method comprising:
   providing a reactant comprising lignin and hydrogen;
   partially deoxygenating the reactant in the presence of a first catalyst at a first temperature, thereby generating an intermediate;
   deoxygenating the intermediate in the presence of a second catalyst at a second temperature, thereby generating a product comprising aromatic hydrocarbons.
Example 2. The method of example 1, wherein the second temperature is greater than the first temperature.
Example 3. The method of example 1 or 2, wherein the reactant comprises greater than 50% lignin oil.
Example 4. The method of any of examples 1-3, wherein the reactant comprises greater than 99% lignin oil.
Example 5. The method of any of examples 1-4, wherein the reactant does not comprise a solvent.
Example 6. The method of any of examples 1-5, wherein the first temperature is selected from the range of 250° C. to 450° C.
Example 7. The method of any of examples 1-6, wherein the second temperature is selected from the range of 275° C. to 475° C.
Example 8. The method of any of examples 1-7, wherein the first catalyst, the second catalyst or both comprise Mo₂C.
Example 9. The method of any of examples 1-8, wherein the first catalyst and the second catalyst are the same.
Example 10. The method of any examples 1-9, wherein the steps of partially deoxygenating and deoxygenating are each performed continuously.
Example 11. The method of examples 1-10, wherein the steps of partially deoxygenating and deoxygenating are part of a continuous process.
Example 12. The method of any of examples 1-11, wherein the product is sustainable aviation or marine fuel.
Example 13. The method of any of examples 1-12, wherein the product comprises an oxygen mole fraction less than or equal to 5%.
Example 14. A reactor comprising:
   a first reaction zone comprising a first catalyst at a first temperature;
   a second reaction zone comprising a second catalyst at a second temperature;
   where in the reactor is capable of performing hydrodeoxygenation of lignin to generate aromatic products.
Example 15. The reactor of example 14, wherein the second temperature is greater than the first temperature.
Example 16. The reactor of example 14 or 15, wherein the reactor is a trickle bed reactor.
Example 17. The reactor of any of examples 14-16, wherein the first catalyst, the second catalyst or both comprise Mo₂C.
Example 18. The reactor of any of examples 14-17, wherein the reactor is capable generating aromatic products with continuous flow.
Example 19. A catalyst comprising Mo₂C, wherein the catalyst is capable of converting lignin to aromatic compounds at a yield greater than or equal to 80%.

The terms and expressions which have been employed herein are used as terms of description and not of limitation, and there is no intention in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments, exemplary embodiments and optional features, modification and variation of the concepts herein disclosed may be resorted to by those skilled in the art, and that such modifications and variations are considered to be within the scope of this invention as defined by the appended claims. The specific embodiments provided herein are examples of useful embodiments of the present invention and it will be apparent to one skilled in the art that the present invention may be carried out using a large number of variations of the devices, device components, methods steps set forth in the present description. As will be obvious to one of skill in the art, methods and devices useful for the present methods can include a large number of optional composition and processing elements and steps.

As used herein and in the appended claims, the singular forms “a”, “an”, and “the” include plural reference unless the context clearly dictates otherwise. Thus, for example, reference to “a cell” includes a plurality of such cells and equivalents thereof known to those skilled in the art. As well, the terms “a” (or “an”), “one or more” and “at least one” can be used interchangeably herein. It is also to be noted that the terms “comprising”, “including”, and “having” can be used interchangeably. The expression “of any of claims XX-YY” (wherein XX and YY refer to claim numbers) is intended to provide a multiple dependent claim in the alternative form, and in some embodiments is interchangeable with the expression “as in any one of claims XX-YY.”

When a group of substituents is disclosed herein, it is understood that all individual members of that group and all subgroups, are disclosed separately. When a Markush group or other grouping is used herein, all individual members of the group and all combinations and subcombinations possible of the group are intended to be individually included in the disclosure. For example, when a device is set forth disclosing a range of materials, device components, and/or device configurations, the description is intended to include specific reference of each combination and/or variation corresponding to the disclosed range.

Every formulation or combination of components described or exemplified herein can be used to practice the invention, unless otherwise stated.

Whenever a range is given in the specification, for example, a density range, a number range, a temperature range, a time range, or a composition or concentration range, all intermediate ranges and subranges, as well as all individual values included in the ranges given are intended to be included in the disclosure. It will be understood that any subranges or individual values in a range or subrange that are included in the description herein can be excluded from the claims herein.

All patents and publications mentioned in the specification are indicative of the levels of skill of those skilled in the art to which the invention pertains. References cited herein are incorporated by reference herein in their entirety to indicate the state of the art as of their publication or filing date and it is intended that this information can be employed herein, if needed, to exclude specific embodiments that are in the prior art. For example, when composition of matter is claimed, it should be understood that compounds known and available in the art prior to Applicant's invention, including compounds for which an enabling disclosure is provided in the references cited herein, are not intended to be included in the composition of matter claims herein.

As used herein, “comprising” is synonymous with “including,” “containing,” or “characterized by,” and is inclusive or open-ended and does not exclude additional, unrecited elements or method steps. As used herein, “consisting of” excludes any element, step, or ingredient not specified in the claim element. As used herein, “consisting essentially of” does not exclude materials or steps that do not materially affect the basic and novel characteristics of the claim. In each instance herein any of the terms “comprising”, “consisting essentially of” and “consisting of” may be replaced with either of the other two terms. The invention illustratively described herein suitably may be practiced in the absence of any element or elements, limitation or limitations which is not specifically disclosed herein.

All art-known functional equivalents, of any such materials and methods are intended to be included in this invention. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

What is claimed is:

1. A method comprising:
providing a reactant comprising lignin and hydrogen;
partially deoxygenating the reactant in the presence of a first catalyst at a first temperature, thereby generating an intermediate;
deoxygenating the intermediate in the presence of a second catalyst at a second temperature, thereby generating a product comprising aromatic hydrocarbons.

2. The method of claim 1, wherein the second temperature is greater than the first temperature.

3. The method of claim 1, wherein the reactant comprises greater than 50% lignin oil.

4. The method of claim 1, wherein the reactant comprises greater than 99% lignin oil.

5. The method of claim 1, wherein the reactant does not comprise a solvent.

6. The method of claim 1, wherein the first temperature is selected from the range of 250° C. to 450° C.

7. The method of claim 1, wherein the second temperature is selected from the range of 275° C. to 475° C.

8. The method claim 1, wherein the first catalyst, the second catalyst or both comprise Mo₂C.

9. The method of claim 1, wherein the first catalyst and the second catalyst are the same.

10. The method of claim 1, wherein the steps of partially deoxygenating and deoxygenating are each performed continuously.

11. The method claim 1, wherein the steps of partially deoxygenating and deoxygenating are part of a continuous process.

12. The method of any claim 1, wherein the product is sustainable aviation or marine fuel.

13. The method of claim 1, wherein the product comprises an oxygen mole fraction less than or equal to 5%.

14. A reactor comprising:
a first reaction zone comprising a first catalyst at a first temperature;
a second reaction zone comprising a second catalyst at a second temperature;
where in the reactor is capable of performing hydrodeoxygenation of lignin to generate aromatic products.

15. The reactor of claim 14, wherein the second temperature is greater than the first temperature.

16. The reactor of claim 14, wherein the reactor is a trickle bed reactor.

17. The reactor of claim 14, wherein the first catalyst, the second catalyst or both comprise Mo₂C.

18. The reactor of claim 14, wherein the reactor is capable generating aromatic products with continuous flow.

19. A catalyst comprising Mo₂C, wherein the catalyst is capable of converting lignin to aromatic compounds at a yield greater than or equal to 80%.
